(19) United States
(12) Patent Application Publication
(10) Pub. No.: US 2019/0282114 A1
(43) Pub. Date: Sep. 19, 2019

(54) MULTI-FUNCTIONAL ADAPTER CLIP

(71) Applicant: Shenzhen Launch Electrical Co., Ltd, Shenzhen (CN)

(72) Inventors: Jiaan Huang, Shenzhen (CN); Shuangshuai Zhou, Shenzhen (CN)

(21) Appl. No.: 16/427,312

(22) Filed: May 30, 2019

Related U.S. Application Data
(63) Continuation of application No. PCT/CN2017/108264, filed on Oct. 30, 2017.

(30) Foreign Application Priority Data
Oct. 25, 2017 (CN) 201721387018.7

(51) Int. Cl. A61B 5/0416; A61B 5/0408

(52) U.S. Cl. CPC A61B 5/0416; A61B 5/04085; A61B 2562/225; A61B 2562/16; A61B 2562/0209

(57) ABSTRACT

The present disclosure relates to a multi-functional adapter clip including a receptacle which is provided with an insertion interface capable of inserting therein a banana plug electrode, a fastening hole capable of fastening a stud electrode, and a clamping portion capable of clamping a sheet electrode. It adopts upper and lower plastic cases, a spring, and metal elastic pieces. The adapter clip can be connected with ϕ 4.0 mm banana plug electrode or a ϕ 3.0 mm pin electrode. It can also be connected with ϕ 4.0 mm stud electrode and can be used as a clip-on electrode. After the clamping portion is clamped together, a boss of the upper plastic case is moved by tension of the spring and pushes a narrow waist portion of the stud electrode into a U-shaped groove in the lower plastic case to lock the stud electrode.

### CROSS REFERENCE TO RELATED APPLICATIONS

The present application is a Continuation Application of PCT Application No. PCT/CN2017/108264 filed on Oct. 30, 2017, which claims the priority of Chinese Patent Application No. 201721387018.7 filed on Oct. 25, 2017. The contents of all of the above are hereby incorporated by reference.

### TECHNICAL FIELD

The present disclosure relates to a component of a medical instrument, and in particular to a multi-functional adapter clip. The multi-functional adapter clip is used as an accessory of an electrocardiogram monitor.

### BACKGROUND

In recent years, multi-functional adapter clips have been widely used in clinical applications as an accessory of electrocardiogram monitors. This product has the advantages that lead wires can be shared, operation is easy, and environment is protected. It is accepted by more and more doctors and patients. There are few similar products on the market at home and abroad. The traditional approach is: the electrocardiogram lead wires that are associated with electrocardiogram monitors are formed into banana plug-type lead wires, snap-type lead wires, and clip-type lead wires. In use, doctors or nurses need to change the lead wires according to the condition of electrodes at the patient's end.

The use of a multi-functional adapter clip is mainly an application of its ease of use characteristics. During the use of lead wires, it is extremely inconvenient for doctors or nurses to change the type of lead wires according to the condition of electrodes at the patient's end. The products currently existing on the market adopt the traditional electrode snap connection, which is the adoption of spring and a male and female fastening system. However, since the fastening force is relatively small, there is a risk of falling off of electrodes due to patient's struggle or activity during clinical use. The present disclosure solves the problem of falling off of electrodes. Furthermore, it can be connected with various types of patient's electrodes.

### SUMMARY

Aiming at the deficiencies in the existing technology, the technical problem to be solved by the present disclosure is to provide a multi-functional adapter clip.

In order to solve the above technical problem, the present disclosure is implemented by the following solutions. A multi-functional adapter clip includes a receptacle which is provided thereon with an insertion interface capable of inserting therein a banana plug electrode, a fastening hole capable of fastening a stud electrode, and a clamping portion capable of clamping a sheet electrode. The receptacle includes an upper plastic case, a spring, a lower plastic case, a metal elastic piece, and a fastening elastic piece. The upper plastic case and the lower plastic case are connected by a connecting shaft. A pressing portion is provided on the upper plastic case.

The fastening hole is provided at a bottom portion of the lower plastic case. The fastening elastic piece is disposed at the fastening hole. The fastening elastic piece disposed at the fastening hole is formed with an opening. A narrow waist portion of the stud electrode is engageable in the opening. A boss is provided on an inner surface of an arc-shaped plate body of the upper plastic case, such that after the clamping portion is clamped together, the boss of the upper plastic case is moved by the tension of the spring, and through the closing up of the clamping portion of the clip, the boss is moved and pushes the narrow waist portion of the stud electrode into a U-shaped groove position in the lower plastic case to lock the stud electrode.

Compared with the existing technology, the beneficial effect of the present disclosure is that the multi-functional adapter clip of the present disclosure is more convenient to use. A user only changes the connection on the adapter clip according to the condition of the electrodes at the patient's end. It is not necessary to change the lead wires.

### BRIEF DESCRIPTION OF THE DRAWINGS

FIG. 1 is a front perspective view of the multi-functional adapter clip according to an embodiment of the present disclosure;

FIG. 2 is an exploded view of FIG. 1;

FIG. 3 is an exploded view of the other side of the multi-functional adapter clip according to an embodiment of the present disclosure;

FIG. 4 is a cross-sectional view of the receptacle according to an embodiment of the present disclosure;

FIG. 5 is a perspective view of the receptacle according to an embodiment of the present disclosure;

FIG. 6 is a schematic view showing the structure of the stud electrode being engaged with the receptacle according to an embodiment of the present disclosure;

FIG. 7 is a schematic side view of the receptacle according to an embodiment of the present disclosure; and

FIG. 8 is a perspective view of one end of the receptacle for connecting to a banana plug electrode according to an embodiment of the present disclosure.

### DETAILED DESCRIPTION OF PREFERRED EMBODIMENTS

The preferred embodiments of the present disclosure will be described in detail below with reference to the accompanying drawings in order to make the advantages and features of the present disclosure more readily understood by those skilled in the art, so that the scope of protection of the present disclosure is more clearly defined.

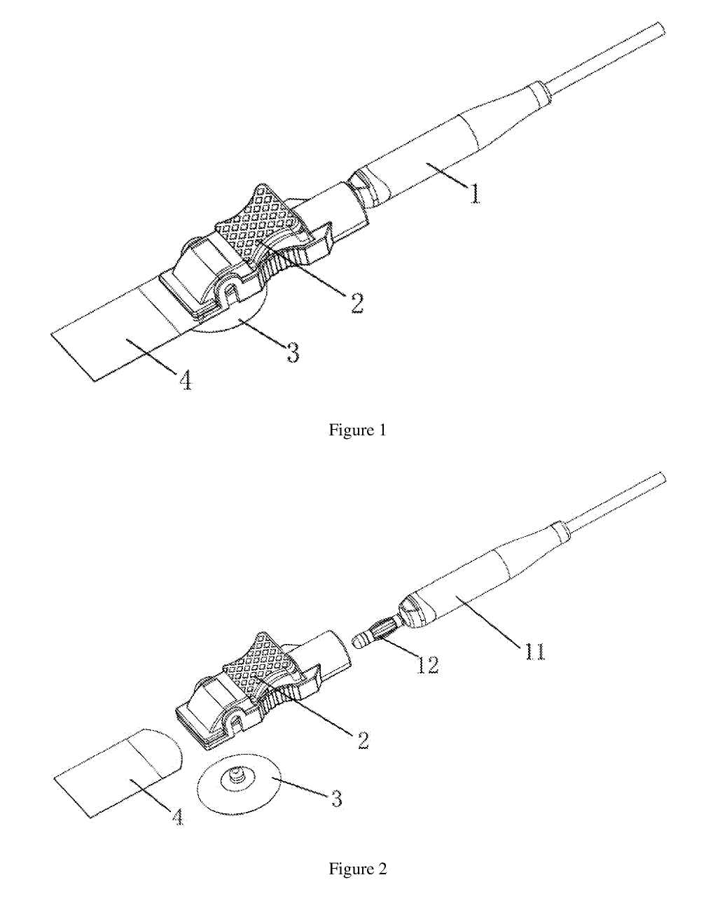

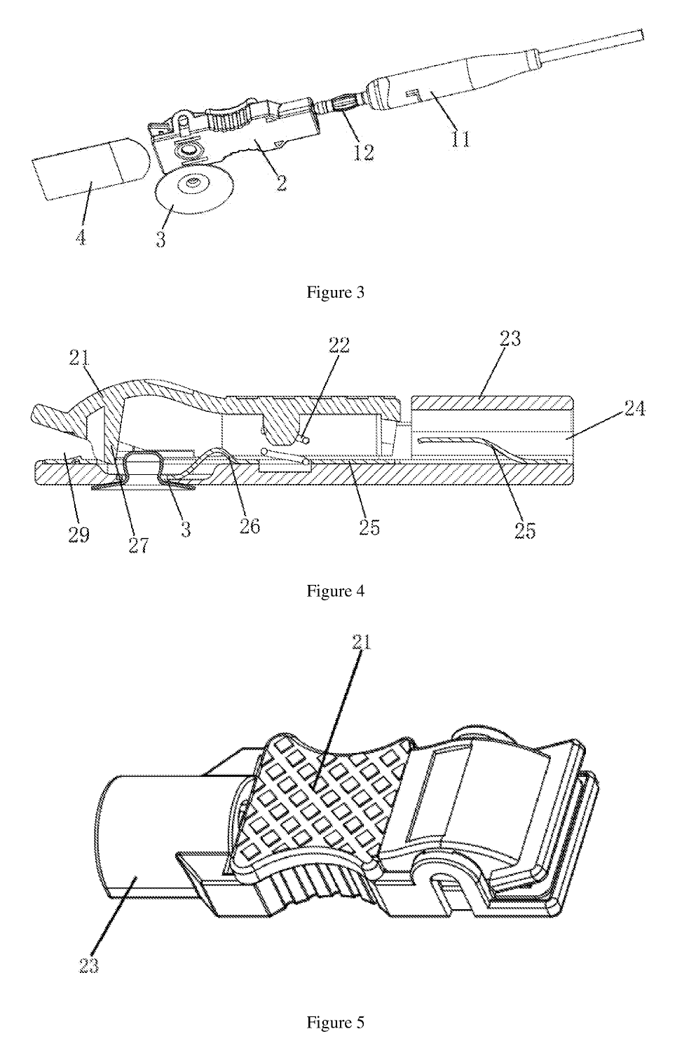

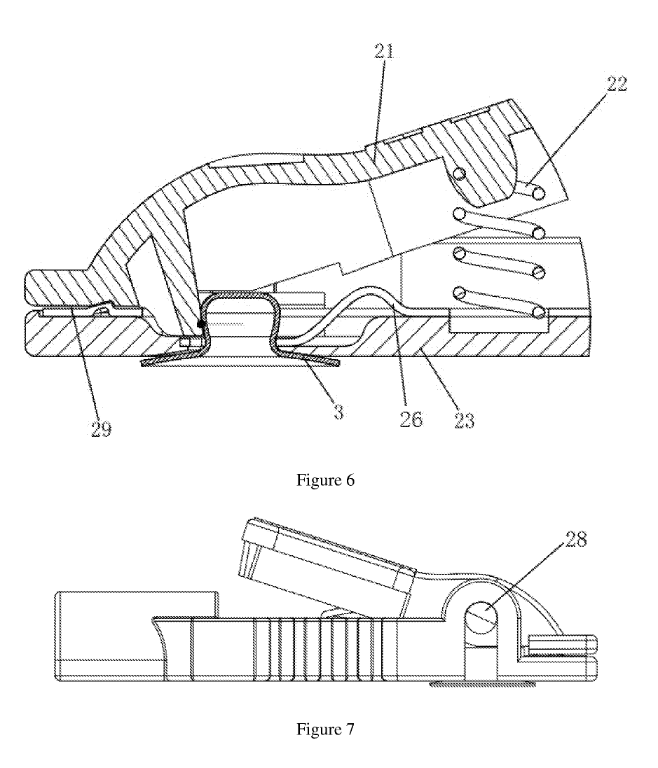

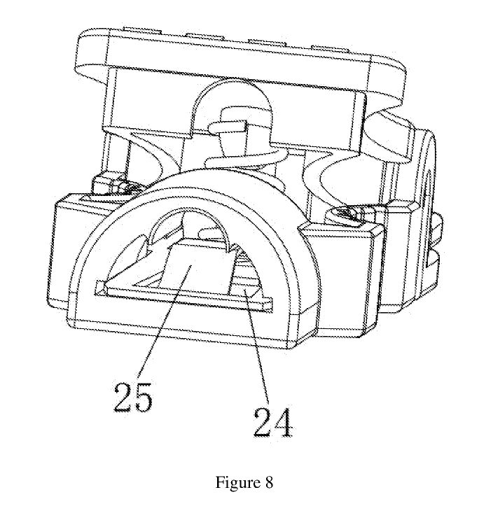

Referring to FIGS. 1-8, a multi-functional adapter clip of the present disclosure may include a receptacle 2. The receptacle 2 may be provided thereon with:

an insertion interface 24 capable of inserting therein a banana plug electrode 1,

a fastening hole capable of fastening a stud electrode 3, and

a clamping portion 29 capable of clamping a sheet electrode 4.

The receptacle 2 may include an upper plastic case 21, a spring 22, a lower plastic case 23, metal elastic pieces 25, and a fastening elastic piece 26.

The upper plastic case 21 and the lower plastic case 23 may be connected by a connecting shaft 28. The upper plastic case 21 may be provided with a pressing portion. The pressing portion may be connected with a plate body extending in a direction towards where the sheet electrode is to be fastened. This plate body may be an arc-shaped plate body. An inner end of the arc-shaped plate body may be connected with a plate body which may be parallel to the lower plastic case 23. A clamping portion may be formed between the plate body and the lower plastic case 23. The clamping portion at the lower plastic case 23 may be provided with a recessed surface. A metal elastic piece 25 may be disposed on the recessed surface. The metal elastic piece 25 disposed on the recessed surface may be provided with a projected portion, which can be used to prevent the sheet electrode 4 from falling off.

An inner side of the pressing portion of the upper plastic case 21 may be provided with a protruding column. One end of the spring 22 may be fixedly connected to the protruding column, and the other end of the spring 22 may be fixed in an indentation formed in the lower plastic case 23.

The fastening hole may be formed at a bottom portion of the lower plastic case 23. A fastening elastic piece 26 may be disposed at the fastening hole. The fastening elastic piece 26 disposed at the fastening hole may be formed with an opening. A narrow waist portion of the stud electrode 3 can be engaged in the opening. A boss 27 may be provided on an inner surface of the arc-shaped plate body of the upper plastic case 21. After the clamping portion is clamped together, the boss 27 of the upper plastic case 21 can be moved by the tension of the spring 22. Through the closing up of the clamping portion of the clip, the boss 27 can be moved so that it can push the narrow waist portion of the stud electrode 3 into a U-shaped groove position in the lower plastic case to lock the stud electrode.

In addition, a metal elastic piece 25 may be provided in the insertion interface 24 on the lower plastic case 23. An inner end of the metal elastic piece 25 in the insertion interface may be bent up into a tongue piece.

The metal elastic piece 25 at the clamping portion and the metal elastic piece 25 at the insertion interface 24 may be connected together to form an integrated metal elastic piece.

The surface of the pressing portion of the upper plastic case 21 may be provided with a raised pattern to increase friction.

The lower plastic case 23, the connecting end of the banana plug electrode 1, and the pressing portion of the upper plastic case 21 may be parallel to each other. The case body where the insertion interface 24 is located may be semi-circular in shape. A top side of the insertion interface 24 may have a semi-circular groove. Two lateral arc-shaped grooves may be formed along the two sides of the semi-circular groove respectively.

The multi-functional adapter clip of the present disclosure may adopt two plastic cases (i.e. the upper plastic case 21 and the lower plastic case 23), tension of the built-in spring 22, and fixed-type metal elastic pieces. After assembly, multiple functions of the product can be realized. It can be connected with ϕ 4.0 mm banana plug electrode or ϕ 3.0 mm pin electrode. It can also be connected with ϕ 4.0 mm stud electrode. It can also be used as a clip-type electrode. Thus, it is more convenient for clinical use. After closing up of the clamping portion of the multi-functional adapter clip and through the tension of the spring 22 and movement of the upper plastic case, the boss of the upper plastic case can be moved and can push the narrow waist portion of the stud electrode into a U-shaped groove position in the lower plastic case to lock the stud electrode. This can prevent the snap stud from falling off during use. As such, its performance is more reliable.

The multi-functional adapter clip of the present disclosure may adopt ϕ 4.0 mm receptacle for connection with ϕ 4.0 mm banana plug electrode, or may adopt ϕ 3.0 mm receptacle for connection with ϕ 3.0 mm pin electrode. It can also replace the clip-on electrode process.

After assembly is completed and when the product is in use, the banana plug or pin 12 with lead wire 11 can be inserted into ϕ 4.0 mm or ϕ 3.0 mm receptacle of the multi-functional adapter clip. It can also be connected with a 4.0 mm stud-type electrode. It can also replace the clip-on electrode. Hence, the multi-functional adapter clip of the present disclosure is more convenient for clinical use.

The above description covers only the preferred embodiments of the present disclosure, and does not limit the scope of patent protection of the present disclosure. Any equivalent structure or equivalent process transformation made by using the specification and the drawings of the present disclosure, or directly or indirectly applied to other related technical fields are similarly within the scope of patent protection of the present disclosure.

What is claimed is:

1. A multi-functional adapter clip, the adapter clip comprising a receptacle, the receptacle being provided thereon with an insertion interface capable of inserting therein a banana plug electrode; a fastening hole capable of fastening a stud electrode; and a clamping portion capable of clamping a sheet electrode, the receptacle comprising an upper plastic case; a spring; a lower plastic case; a metal elastic piece; and a fastening elastic piece, the upper plastic case and the lower plastic case being connected by a connecting shaft, and a pressing portion provided on the upper plastic case,
wherein the fastening hole is provided at a bottom portion of the lower plastic case, the fastening elastic piece is disposed at the fastening hole, the fastening elastic piece disposed at the fastening hole is formed with an opening, and a narrow waist portion of the stud electrode is engageable in the opening, and a boss is provided on an inner surface of an arc-shaped plate body of the upper plastic case, such that after the clamping portion is clamped together, the boss of the upper plastic case is moved by the tension of the spring, and through the closing up of the clamping portion of the clip, the boss is moved and pushes the narrow waist portion of the stud electrode into a U-shaped groove position in the lower plastic case to lock the stud electrode.
